(19) United States
(12) Patent Application Publication
(10) Pub. No.: US 2014/0328956 A1
(43) Pub. Date: Nov. 6, 2014

(54) Use Of Two Botanicals With Complementary Activities For Improvement Of Skin

(71) Applicant: The Regents of the University of Michigan, Ann Arbor, MI (US)

(72) Inventors: James Varani, Ann Arbor, MI (US); Kent J. Johnson, Ann Arbor, MI (US)

(21) Appl. No.: 14/334,036

(22) Filed: Jul. 17, 2014

Related U.S. Application Data
(62) Division of application No. 12/605,431, filed on Oct. 26, 2009.
(60) Provisional application No. 61/197,406, filed on Oct. 27, 2008.

(51) Int. Cl. A61K 31/12; A61K 36/9066; A61K 36/9068

(52) U.S. Cl. CPC A61K 31/12; A61K 36/9068; A61K 36/9066
USPC 424/756; 514/678

(57) ABSTRACT

A skin augmentation composition comprises a therapeutically effective amount of a combination of a gingerol and a curcumin and a cosmetically or pharmaceutically acceptable carrier. Methods for enhancing the repair of damaged skin and the prevention of developing wounds in a subject having damaged skin comprises administering to a portion of damaged skin, a composition comprising a therapeutically effective amount of a combination of a gingerol and a curcumin and a cosmetically or pharmaceutically acceptable carrier.

### CROSS-REFERENCE TO RELATED APPLICATIONS

This application claims the benefit of U.S. Provisional Application No. 61/197,406, filed on Oct. 27, 2008. The entire disclosure of the above application is incorporated herein by reference.

### FIELD

The present technology relates to botanical compositions for the treatment and improvement of skin structure and function for the purpose of enhancing the capacity of the skin to repair abrasion wounds that subsequently occur.

### INTRODUCTION

This section provides background information related to the present technology, which is not necessarily prior art.

The connective tissue of the skin deteriorates as a consequence of the natural aging process. There is less intact collagen and an increase in fragmented collagen in skin from individuals aged 80+ years as compared to individuals aged 18-29 years. Collagen damage is exacerbated in areas of photodamage (skin that has been chronically exposed to UV radiation from sun over years). Exposure to sunlight is such a pronounced factor in premature aging that by middle age individuals who have been exposed to more sunlight appear older than those who have not. The extent of dermal degenerative change correlates with the visible signs of premature aging. The subepidermal band of normal dermis, which is a site of continual dermal repair, contains normal collagen fibers. This zone becomes visually evident, however, only after there is sufficient elastotic damage to delineate this region. The elastotic material is composed principally of elastin and microfibrillar proteins that codistribute with fibronectin (see, Schwartz (1988) J. Invest. Dermatol. 1:158-161). Actinic elastosis appears to be reversible to some extent by treatment with chemical peels, dermabrasion or topical application of tretinoin (see, e.g., Warren et al. (1991) J. American Acad. Dermatol. 25:751-760; and Weiss et al. (1988) J. American Medical Assoc. 259:527-532).

Extended use of cortico steroids causes atrophy in the skin as these agents dramatically inhibit collagen synthesis. Metabolic diseases such as diabetes also lead to severe damage to collagen in the skin. In all of these conditions, collagen synthesis is decreased, and collagen-degrading matrix metalloproteinases (MMPs) are increased. Skin that has severe connective tissue deficits is prone to bruising. When damaged skin suffers minor abrasion-type injuries, the wounds heal more slowly. Often, such wounds do not heal at all, but go on to form chronic ulcers with devastating consequences.

Superficial wounds that occur in healthy skin are expected to heal without incident. In contrast, wounds in chronically-damaged or atrophic skin heal more slowly and may form non-healing ulcers. Although multiple factors contribute to impaired wound healing, damage to the underlying connective tissue is a major impediment to efficient wound closure. Recent studies have shown that when there is extensive damage to type I collagen in the dermis (the major connective tissue element in the dermal matrix), dermal fibroblasts assume a degradative phenotype. Under such conditions, production of connective tissue elements is reduced and MMP levels are increased. Fibroblasts are not alone in their response to the damaged connective tissue. Other studies have demonstrated that the underlying dermal matrix influences keratinocyte migration. Efficient migration requires an intact matrix, which the keratinocytes, themselves, fragment during migration. The fragmented matrix does not support keratinocyte adhesion as efficiently as the intact matrix. Based on these observations, it is believed that poor skin wound healing in aged individuals (as well as in people with diabetes, on corticosteroid therapy, etc.) reflects connective tissue damage rather than defects in the cellular elements, per se.

All-trans retinoic acid (RA) is the only agent currently approved by the FDA for the purpose of skin-repair, and in past studies, RA and other retinoids have been shown to improve wound healing in damaged or atrophic skin. Improved wound healing is associated with increased collagen production and/or decreased breakdown. While retinoid use might provide beneficial effects in damaged skin, these agents cannot, ultimately, be optimal since in most users, retinoids are irritating to the skin.

Clinical studies have documented improved appearance of aged and photoaged skin following topical retinoid use. Increased production of type I procollagen and decreased elaboration of several MMPs accompany improved appearance. Not surprisingly, a number of studies have documented beneficial effects of RA and other biologically-active retinoids in wound healing models.

Long-term ultraviolet exposure results in histological and visible changes in the skin, including: damage to the underlying connective tissue, manifested as elastosis and increases in the glycosaminoglycans and loss of collagen; dermal accumulation of elastin-staining material resulting from the degenerative changes in collagen fibers; epidermal dysplasia with cytologic atypia and loss of polarity of keratinocytes; and an inflammatory infiltrate (see, e.g., Bissett et al. (1987) Photochemistry and Microbiology 46:367-378). The degradation of elastic fibers and wrinkling associated with intrinsically aging skin also accompanies photoaging. In humans, advanced photodamage can be detected in the staining properties of dermal tissue resulting from changes in the insoluble and soluble fractions of collagen that occur as the entire upper dermis becomes filled with elastosis (Kligman et al. (1989) J. Investigative Dermatol. 93:210-214). The changes in collagen and elastic fiber over decades of such exposure result in skin that is wrinkled, yellowed, blotchy, lax, rough and leathery. Scanning electron microscopy of aged skin indicates that the network of elastic fibers becomes denser and has a more disorganized arrangement than younger skin.

While prophylactic use of RA as a wound damage preventative is an attractive strategy for individuals with severely damaged skin, topical retinoid use elicits skin irritation in most individuals. Skin irritation is the major reason for noncompliance among retinoid users. Further, if irritation is too great, the inflammatory changes can counteract the beneficial effects on collagen metabolism or even increase the susceptibility of the skin to wounding. Identification of other agents with skin-repair potential that can enhance the capacity of the skin to repair abrasion wounds would be beneficial.

### SUMMARY

This section provides a general summary of the disclosure, and is not a comprehensive disclosure of its full scope or all of its features.

The present technology provides for a skin augmentation composition comprising curcumin, 6-gingerol, and a carrier suitable for topical application. The composition may include from about 1% to about 20% curcumin (w/v) and/or may include from about 0.1% to about 10% 6-gingerol (w/v). For example, the composition may include about 10% curcumin (w/v) and about 3% 6-gingerol (w/v). Analogues and derivatives of curcumin and 6-gingerol, including more than one type of each, may also be used. The curcumin and/or 6-gingerol may be synthetic or may be derived from one or more plant extracts, including turmeric (Curcuma longa) and ginger (Zingiber officinale).

Other aspects of the present technology include methods for enhancing the repair of damaged skin and the prevention of developing wounds in a subject having damaged skin. Such methods can include administering to a portion of damaged skin, a composition comprising a therapeutically effective amount of a combination of curcumin, 6-gingerol, and a carrier suitable for topical application. Methods further include treating the chronological aging of skin and treating photoaged skin with the present compositions. In some cases, a method for enhancing the capacity of skin to repair a wound includes administering to the skin a first composition comprising curcumin and a carrier suitable for topical application and administering a second composition comprising 6-gingerol and a carrier suitable for topical application. Administration of one of the first and second compositions may occur within less than about one day from the administration of the other one of the first and second compositions.

Further areas of applicability will become apparent from the description provided herein. The description and specific examples in this summary are intended for purposes of illustration only and are not intended to limit the scope of the present technology.

### DRAWINGS

The drawings described herein are for illustrative purposes only of selected embodiments and not all possible implementations, and are not intended to limit the scope of the present technology.

FIG. 1 shows photographs of wounded skin of hairless rats treated with a control vehicle, curcumin (10% w/v), ginger extract (3% w/v) and a combination of curcumin and ginger demonstrates histological features seen in the wounded and healed skin from representative animals.

FIG. 2 shows a bar chart describing the effect of the various treatments on skin healing versus time. Abrasion wound healing in control hairless rats and hairless rats treated with Temovate® (clobetasol propionate) plus vehicle, curcumin, ginger extract or curcumin and ginger extract. Values represent the percentage of the wound that is closed and scab-free at each time-point. Values are based on nine animals per group. Values from the three groups were evaluated statistically (ANOVA followed by paired group comparisons).

FIG. 3 shows a bar chart representing the levels of collagen produced by skin in an organ culture after treatment of the wounded skin with vehicle alone, Temovate® (Tem) plus vehicle, curcumin (Cur), ginger extract, and curcumin and ginger extract. Soluble collagen type 1 in organ culture fluid of skin from control rats and from rats treated with Temovate® plus vehicle, curcumin, ginger extract or curcumin and ginger extract is determined by Western blotting. The biopsy was taken from an area within the initial abrasion wound margin. Values are means and standard deviations based on organ cultures from nine rats per treatment group. Insert shows an electropherogram of representative western blots of organ culture fluid from each of the treatment groups.

FIG. 4 shows a bar graph illustrating the levels of MMP-2 and MMP-9 (gelatin zymography) in organ culture fluid of skin from control rats and from rats treated with Temovate® plus vehicle, curcumin, ginger extract, or curcumin and ginger extract. The biopsy was taken from an area within the initial abrasion wound margin. Values are means and standard deviations based on organ cultures from nine rats per treatment group. Values were analyzed for statistical significance using the Student T-test, comparing each site separately. Insert: shows an electropherogram of representative gelatin zymograms of organ culture fluid from each of the treatment groups.

FIG. 5 shows a bar graph measuring the numbers of fibroblasts after incubation under control conditions (KBM culture medium) or in the same culture medium supplemented with 2.5 μM curcumin, 0.5 μg per mL ginger extract or the combination of the two. Only the combination resulted in a significant and synergistic increase in cell number over that of the control.

FIG. 6 shows a bar graph representing the relative levels of MMP-1 (Y-axis) in human skin organ culture fluid after incubation with curcumin and curcumin with ginger (X-axis). Human skin was incubated in organ culture under control conditions (Ca²⁺—supplemented KBM culture medium) or in the same culture medium with 1 μg per mL ginger extract. Increasing amounts of curcumin were added. As can be seen from the figure, the ginger extract induced MMP-1 expression and this was inhibited by curcumin.

FIG. 7 shows a bar graph representing the relative levels of MMP-1 (Y-axis) in fibroblast culture fluid. Fibroblasts were incubated under control conditions (Ca²⁺-supplemented KBM culture medium) or in the same culture medium supplemented with 1 μg per mL ginger extract. Increasing amounts of curcumin (X-axis) were added. As can be seen from the figure, the ginger extract induced MMP-1 expression and this was inhibited by curcumin.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Detailed Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the apparatus and systems of this technology and, unless explicitly stated otherwise, are not intended to be a representation that given embodiments of this technology have, or have not, been made or tested.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. In addition, disclosure of ranges includes disclosure of all distinct values and further divided ranges within the entire range.

The present technology relates to compositions and methods that include curcumin and gingerol compounds. A skin augmentation composition is provided that comprises active curcumin and gingerol compounds and/or physiologically active derivatives and homologs thereof, and methods for using these compositions for the augmentation or reparation of connective tissue deficits in skin that has been damaged due to several risk factors, including chronological aging, chronic damage due to diabetes, photoaging and extended use of cortico steroids.

Skin augmentation compositions for the treatment and prevention of atrophic and damaged skin can include the following aspects. The skin augmentation composition of the present invention can contain a safe and effective amount of at least one curcumin compound and at least one gingerol compound. In some embodiments of the present technology, a combination of two natural agents—curcumin (e.g., from tumeric) and 6-gingerol (e.g., obtained from an enriched extract of ginger root)—improves collagen metabolism and improves healing of superficial abrasion wounds that are subsequently induced in photodamaged, atrophic, or corticosteroid-treated (at-risk) skin. This is accomplished in the absence of significant skin irritation. The active compounds may be isolated from natural botanical extracts, may be synthetic, and may include mixtures derived from natural and synthetic sources.

The first active agent of the present skin augmentation composition includes curcumin. Curcumin can be derived from the Indian spice turmeric (Curcuma longa). Tumeric contains three major curcuminoids, namely, curcumin, demethoxycurcumin, and bisdemethoxycurcumin. Curcumin is the principal curcuminoid in tumeric, being the yellow color pigment in turmeric, and can be produced industrially from turmeric oleoresin. The formulaic name for curcumin to be used as an active ingredient in the present skin augmentation composition is (1E,6E)-1,7-bis(4-hydroxy-3-methoxyphenyl)hepta-1,6-diene-3,5-dione, which is represented by the following structure:

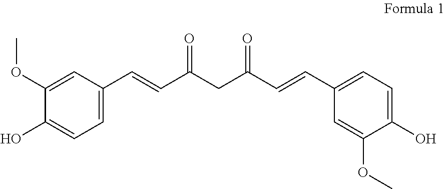 [SMILES: COc1cc(/C=C/C(=O)CC(=O)/C=C/c2ccc(O)c(OC)c2)ccc1O]

In some embodiments, one or more analogs of curcumin can also be used as the curcumin active or in addition to curcumin, as shown in formula 1. Analogs of curcumin include: (1E,4Z,6E)-5-hydroxy-1,7-bis(4-hydroxy-3-methoxyphenyl)hepta-1,4,6-trien-3-one,1,7-bis(4-hydroxy-3 methoxyphenyl)hepta-1,6-diene-3,5-dione; (1E,4Z,6E)-1,7-dideuterio-5-hydroxy-1,7-bis(4-hydroxy-3-methoxyphenyl)hepta-1,4,6-trien-3-one; (1E,4Z,6E)-5-hydroxy-1,7-bis(4-hydroxy-3-methoxyphenyl)hepta-1,4,6-trien-3-one; 5-hydroxy-1,7-bis(4-hydroxy-3-methoxyphenyl)hepta-1,4,6-trien-3-one; [4-[(1E,6E)-7-(4-acetyloxy-3-methoxyphenyl)-3,5-dioxohepta-1,6-dienyl]-2-methoxyphenyl]acetate; (2E,7E)-1,9-bis(4-hydroxyphenyl)nona-2,7-diene-4,6-dione; (1E,6E)-1,7-bis(3-methoxy-4-prop-2-enoxyphenyl)hepta-1,6-diene-3,5-dione; (1E,6E)-1,7-bis(4-hydroxyphenyl)hepta-1,6-diene-3,5-dione, [(2R)-2,5,7,8-tetramethyl-2-[(4R,8R)-4,8,12-trimethyltridecyl]chroman-6-yl]; [2-methoxy-4-[(1E,6E)-7-[3-methoxy-4-[4-oxo-4-[(2R)-2,5,7,8-tetramethyl-2-[(4R,8R)-4,8,12-trimethyltridecyl]chroman-6-yl]oxybutanoyl]oxyphenyl]-3,5-dioxohepta-1,6-dienyl]phenyl]butanedioate; and (1E,4Z,6E)-1,7-bis(3,4-dimethoxyphenyl)-5-hydroxyhepta-1,4,6-trien-3-one. The purity of the curcumin active used in the present skin augmentation composition may be greater than about 90%, greater than about 92%, greater than about 94%, greater than about 96%, greater than about 98%, or at least 99.5% pure.

Methods for isolating and extracting curcumin from the rhizomes of the turmeric plant (Curcuma longa, Zingiberaceae) are well known and can include steam distillation, solvent extraction; e.g., Soxhlet extraction with toluene followed by concentration and slow crystallization and supercritical fluid extraction using CO₂. Alternatively, curcumin for use in the present compositions and methods can be prepared by synthetic methods. Curcumin is also commercially available from LKT Laboratories, Inc. (St. Paul, Minn.).

In some embodiments, a safe and effective amount of curcumin and/or one or more of its analogs in the skin augmentation composition of the present technology can range from about 1% to about 20% (w/v). In some embodiments, the amount of curcumin in the topical composition is at least about 5%, 7%, 9%, 11%, 13%, 15%, 17% or at least about 19% (% w/v). In some embodiments, the relative amount of curcumin and/or one or more of its analogs in the topical composition of the present technology is at least about 10%.

The skin augmentation composition of the present technology includes a safe and effective amount of a second active agent, 6-gingerol, a phenolic compound found in the root of the ginger plant (Zingiber officinale, Roscoe, Zingiberaceae). The ginger root is also a source of multiple biologically-active compounds, including 6-gingerol, 8-gingerol, 10-gingerol, 6-paradol, 6-shagoal and cassumunin. 6-gingerol is typically the most abundant analog in an extract, and may represent approximately 32% of the total extract. Methods for extracting 6-gingerol from the ginger root are well known and can include steam distillation, solvent extraction and supercritical fluid extraction using CO₂. 6-gingerol is also commercially available from Dalton Chemical Laboratories (Toronto, Canada).

6-gingerol has the formulaic name of 5-hydroxy-1-(3-hydroxy-4-methoxyphenyl)decan-3-one and is represented by the following structure

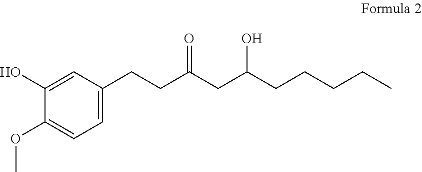 [SMILES: CCCCCC(O)CC(=O)CCc1ccc(OC)c(O)c1]

The skin augmentation composition of the present technology contains a safe and effective amount of 6-gingerol. Other impurities contained with the 6-gingerol component can include 8-gingerol, 10-gingerol, 6-paradol, 6-shagaol and cassumunin. The 6-gingerol active agent can be in the form of a ginger extract containing a percentage of 6-gingerol ranging from 20% to about 50% (% w/v). In some embodiments, the 6-gingerol has a purity of at least 30%, at least 40%, at least 50%, at least 60%, at least 70%, at least 80%, at least 90% at least 95%, about at least 96%, of about at least 97%, of about at least 98%, of about at least 99%, or at least 99.5% pure.

The amount of 6-gingerol in the skin augmentation composition of the present technology can range from about 0.1% to about 10% (w/v). In some embodiments, the amount of 6-gingerol in the topical composition is at least 0.1%, at least 0.5%, at least 1%, at least 2%, at least 3%, at least 4%, at least 5%, at least 6%, at least 7%, at least 8% or at least 9% (% w/v). In some embodiments, the relative amount of 6-gingerol in the topical composition of the present technology is at least 1%.

The skin augmentation composition can include an extract of ginger and/or a synthetic mixture that can include one or more of 6-gingerol, 8-gingerol, 10-gingerol, 6-paradol, 6-shagaol, and cassumunin. The present technology includes a skin augmentation composition that includes a ginger compound in an amount ranging from 5% to about 30% (w/v of the total composition). The ginger compound can contain an amount of 6-gingerol ranging from about 20% to about 50% (w/v).

There are several surprising and unexpected ways in which the combination of curcumin and 6-gingerol functions. First, these compounds promote collagen building in combination. On one hand, the ginger compound serves to raise the level of collagen-degrading enzymes, such as matrix metalloproteinases (MMPs), as one of its functions. On the other hand, the curcumin compound suppresses the MMP elevation stimulated by the ginger compound. Second, the combination promotes vascularization. During wound healing there is the need for new vascularization. However, new vessel growth needs to halt at some point and some vessels need to regress at a time when the wounded skin is fully healed. The ginger compound stimulates new vessel formation and the combination of curcumin and ginger extract result in a reduction in the number of capillaries in the skin at the end of the wound healing. This allows the skin to return to quiescence. Third, the combination promotes epidermal proliferation. Skin irritation is intimately associated with epidermal hyperplasia. The ginger compound stimulates epidermal keratinocyte proliferation while the curcumin compound suppresses ginger extract-induced keratinocyte growth. This may be the underlying factor responsible for the absence of irritation by the combination, for example, as compared to irritation observed with retinoid therapy. And fourth, curcumin applied alone imparts a yellow-red color to the skin. However, this discoloration is not observed or greatly mitigated in the combination of curcumin and ginger compounds. Also, the ginger compound can have a strong odor if applied alone, but when used in combination, the presence of curcumin lessens the odor. Thus, the curcumin and ginger compounds act together in a synergistic fashion to produce a more effective composition to enhance the capacity of skin to repair wounds, where the combination is also more aesthetically pleasing following application to the skin and has a reduced odor.

In some embodiments, the active agents can be combined with a carrier in the form of powders, gels, liquids or combinations thereof. In some embodiments, the active agents curcumin and 6-gingerol can be mixed in ratios ranging from 10:1 to 1:10 in an appropriate carrier solvent. In some embodiments, the actives can be dissolved or mixed with a solvent comprising 70% ethanol/30% propylene glycol. The skin augmentation compositions of the present technology can comprise the actives, curcumin and 6-gingerol, mixed in a topical formulation with a carrier, such as an excipient comprising 70% ethanol/30% propylene glycol and applied to the skin of the intended subject.

In some embodiments, additional or optional topical skin formulation excipients which are cosmetically and/or pharmaceutically acceptable excipients known to those skilled in the art can be included into the composition of the present technology. Some of the optional excipients that can be admixed into the composition can include one or more of the following: an emollient, a binder, a thickening agent, a filler, a humectant, and the like. Other optional components can include one or more of: a preservative, a modifier, a chelating agent, a fragrance, an antibiotic, an antioxidant and an anti-inflammatory. In the practice of the present technology, it is generally preferred to use water, which has been purified by processes such as deionization, or reverse osmosis, to prevent batch-to-batch formulation inconsistencies, which can be caused by dissolved solids in the water supply. The amount of water in the skin augmentation composition can range from about 50 weight percent to about 94 weight percent, preferably from about 55 to 90 percent, most preferably from about 60 percent to about 90 percent.

An emollient is an oleaginous or oily substance, which helps to smooth and soften the skin, and can also reduce its roughness, cracking or irritation. Typical suitable emollients useful in the formulation of the present skin augmentation composition can include one or more of: mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. In some embodiments, the emollient is a cocoglyceride, which is a mixture of mono, di- and triglycerides of cocoa oil, sold under the trade name of Myritol 331 from Henkel KGaA, or Dicaprylyl Ether available under the trade name Cetiol OE from Henkel KGaA, or a C₁₂-C₁₅ alkyl benzoate sold under the trade name Finsolv™ from Finetex. Another suitable emollient is DC 200 Fluid 350, a silicone fluid, available Dow Corning Corp. (Midland, Mich.). One or more emollients can be present ranging in amounts from about 1 percent to about 10 percent by weight, preferably about 5 percent by weight.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures can be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more of the various emollients can optionally be included in the formulation.

One or more humectants can optionally be included in the formulation in amounts from about 1 percent to about 10 percent by weight, preferably about 5 percent by weight. A humectant is a moistening agent that promotes retention of water due to its hygroscopic properties. Suitable humectants include glycerin, polymeric glycols such as polyethylene glycol and polypropylene glycol, mannitol and sorbitol. In some embodiments, the humectant is sorbitol, 70% USP or polyethylene glycol 400, NF.

A dry-feel modifier is an agent which when added to an emulsion, imparts a “dry feel” to the skin when the emulsion dries. Dry feel modifiers useful in the present compositions can include talc, kaolin, chalk, zinc oxide, silicone fluids, inorganic salts such as barium sulfate, surface treated silica, precipitated silica and fumed silica.

It can be advantageous to incorporate additional thickening agents, for example, various Carbopols™ available from the B. F. Goodrich Co. Illustrative examples can include non-ionic thickening agents. The selection of additional thickening agents is well within the skill of one in the art.

An antimicrobial preservative is a substance or preparation which destroys, or prevents or inhibits the proliferation of, microorganisms in the sunscreen composition, and which can also offer protection from oxidation. Preservatives are frequently used to make self-sterilizing, aqueous based products such as emulsions. This is done to prevent the growth of microorganisms that can be in the product during manufacturing and distribution of the product and during use by consumers, who can further inadvertently contaminate the products during normal use. Typical preservatives can include the lower alkyl esters of para-hydroxybenzoates (parabens), especially methylparaben, propylparaben, isobutylparaben and mixtures thereof, benzyl alcohol, phenyl ethyl alcohol and benzoic acid, diazolydinyl, urea, chlorphenesin, iodopropynyl and butyl carbamate. One or more antimicrobial preservatives can optionally be included in an amount ranging from about 0.001 to about 10 weight percent, preferably about 0.05 to about 1 percent.

An “antioxidant” is a natural or synthetic substance added to the topical composition to protect from or delay its deterioration due to the action of oxygen in the air (oxidation). Although not wishing to be bound by theory, it is believed that the skin augmentation composition of the present technology imparts antioxidant activity to the skin tissue, which reduces the quantity of oxygen reactive species, which are known to cause incidental tissue damage. The present skin augmentation composition can optionally contain an additional antioxidant agent capable of providing additional antioxidant activity to reduce oxidation reactions in skin tissue. Typical suitable antioxidants include propyl, octyl and dodecyl esters of gallic acid, butylated hydroxyanisole (BHA, usually purchased as a mixture of ortho and meta isomers), butylated hydroxytoluene (BHT), green tea extract, uric acid, cysteine, pyruvate, nordihydroguaiaretic acid, Vitamin A, Vitamin E and Vitamin C and their derivatives. One or more antioxidants can optionally be included in the skin augmentation composition in an amount ranging from about 0.001 to about 5 weight percent, preferably about 0.01 to about 0.5 percent.

“Chelating agents” are substances used to chelate or bind metallic ions, such as with a heterocylic ring structure so that the ion is held by chemical bonds from each of the participating rings. Suitable chelating agents include ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, or combinations of any of these.

“Fragrances” can include any substances which can impart an aesthetically pleasing aroma to the topical composition without affecting the activity of the active compounds. Typical fragrances can include aromatic materials extracted from botanical sources (i.e., rose petals, gardenia blossoms, jasmine flowers, etc.) which can be used alone or in any combination to create essential oils. Alternatively, alcoholic extracts can be prepared for compounding fragrances. However, due to the relatively high costs of obtaining fragrances from natural substances, the modern trend is to use synthetically prepared fragrances, particularly in high-volume products. One or more fragrances can optionally be included in the composition in an amount ranging from about 0.001 to about 5 weight percent, preferably about 0.01 to about 0.5 percent by weight.

A “pH modifier” is a compound that will adjust the pH of a formulation to a more acidic pH value or to a more basic pH value. The selection of a suitable pH modifier is well within the ordinary skill of one in the art. Advantageously, a skin augmentation composition according to the present technology will include excipients suitable for administration to the targeted skin area, or can be injected just below the epidermis, or the skin augmentation composition can be administered orally.

The composition can include one or more carriers, including cosmetically or pharmaceutically acceptable excipients which can consist of water or of at least one solvent chosen from hydrophilic organic solvents, lipophilic organic solvents, amphiphilic organic solvents and mixtures thereof, in particular a mixture of water and at least one of the abovementioned solvents.

For topical application, the skin augmentation composition can include a carrier and be in the form of an aqueous, aqueous-alcoholic or oily solution or of a dispersion of the lotion or serum type, of emulsions of liquid or semiliquid consistency of the milk type, which are obtained by dispersing a fatty phase in an aqueous phase (0/W) or conversely (W/O) or multiple emulsions, of a loose or compact powder to be used as it is or to be incorporated into a physiologically acceptable medium, or of suspensions or emulsions of a soft consistency of the aqueous or anhydrous cream or gel type, or alternatively of microcapsules or microparticles, or of vesicular dispersions of the ionic and/or nonionic type. It can also be provided in the form of a salve, a tincture, a cream, an ointment, a powder, a patch, an impregnated pad, a solution, an emulsion or a vesicular dispersion, a lotion, a gel, a spray, a suspension, a shampoo, an aerosol or a foam. It can be anhydrous or aqueous. It can also consist of solid preparations constituting cleansing soaps or cakes. These compositions are prepared according to the customary methods.

For a skin augmentation composition for use topically or for injection, the composition can be provided in the form of an aqueous lotion or an oily suspension. For use orally, the composition can be provided in the form of capsules, granules, syrups to be taken orally or tablets. The quantities of the various constituents of the compositions which can be used according to the invention are those conventionally used in the fields considered. The aqueous phase contains water and optionally an ingredient which is miscible in any proportion with water such as C₁ to C₈ lower alcohols such as ethanol, isopropanol, polyols such as propylene glycol, glycerol, sorbitol, or acetone or ether.

When the skin augmentation composition is an emulsion, the proportion of the fatty phase can range from about 2% to about 80%, in particular from about 5% to about 80% by weight, and preferably from about 5% to about 50% by weight relative to the total weight of the skin augmentation composition. The oils, waxes, emulsifiers and coemulsifiers used in the skin augmentation composition in the form of an emulsion are chosen from those conventionally used in the cosmetic field. The emulsifier and the coemulsifier can be present in the skin augmentation composition in a proportion ranging from about 0.1%, in particular from about 0.3% to about 30% by weight, preferably from about 0.5 to about 20% by weight, and even better from about 1 to about 8% relative to the total weight of the composition. The emulsion can additionally contain lipid vesicles and in particular liposomes. When the skin augmentation composition is an oily solution or gel, the fatty phase can represent more than about 70% of the total weight of the composition. Advantageously, the skin augmentation composition can include microspheres, nanospheres, liposomes, oleosomes or nanocapsules, into which at least one curcumin active agent and at least one 6-gingerol active agent will be encapsulated. The nanospheres can be provided in the form of an aqueous suspension.

The skin augmentation composition including the active agents curcumin and 6-gingerol can also be encapsulated into nanocapsules consisting of a lamellar coating obtained from a silicone surfactant, the nanocapsules can also be prepared based on water-dispersible sulphonic polyesters. In one embodiment, the formulation further includes a surfactant to assist with cell penetration of the active agents or the formulation can contain any suitable loading agent. Any suitable non-toxic surfactant can be included, such as DMSO. Alternatively a transdermal penetration agent such as urea can be included.

Advantageously, for skin application, the composition is an aqueous, alcoholic or aqueous-alcoholic solution or suspension, and preferably a water/ethanol solution or suspension. The alcoholic fraction can represent from about 5% to about 99.9% and even better from about 8% to about 80% of the composition.

Methods for improving the structure and function of skin in subjects at risk for developing slow healing wounds can include the following aspects. Treating and augmenting skin, involves topically applying to the skin, a safe and effective amount of a skin augmentation composition of the present technology. The amount of the composition that is applied, the frequency of application, and the period of use may vary widely depending upon the level of the curcumin and gingerol compounds in a given composition and the level of effect desired, for example, in light of the level of skin damage present or expected to occur.

In some embodiments, the skin augmentation composition is chronically applied to the skin. By “chronic topical application,” it is meant continued topical application of the composition over an extended period during the subject's lifetime, for example, for a period of at least one day, or for a period of at least about one month, or for at least about three months, or for at least about six months, or for at least about one year. While benefits are obtainable after various periods of use (e.g., one month to one, five, ten or twenty years), chronic application can continue throughout the subject's lifetime. Typically applications would be on the order of about once per day over such extended periods, however application rates can vary from about once per week up to about three times per day or more.

Methods further include enhancing the capacity of skin to repair a wound by administering to the skin a composition comprising curcumin, 6-gingerol, and a carrier suitable for topical application. In some cases, the administering is performed prior to the wound formation. The skin may also be treated with a corticosteroid before or after administering the composition. The methods may be used for various wounds, such as a skin abrasion or a surgical incision. For example, the wound may be the recipient site of a skin graft, where the composition is administered before and/or after skin graft is in place. Or, the wound may be the donor site for a skin graft, where the composition is administered to enhance and augment the donor skin prior to transplantation and/or to enhance the capacity of the donor site for repair.

The effective dosage of each of the curcumin and 6-gingerol active compounds employed in the methods and compositions of the present technology can vary depending on a number of factors including the particular concentration of each of the active agents, i.e. curcumin and 6-gingerol, the mode of administration, the frequency of administration, the skin being treated, the type and degree of damage of the skin being treated, the route of administration, the needs of a patient sub-population to be treated or the needs of the individual patient which different needs can be due to age, sex, body weight, relevant dermatological condition specific to the patient. A suitable dose can be from about 0.001 to about 1 mg/kg body weight of each active ingredient, such as about 0.01 to about 0.4 mg/kg body weight of each active agent. As used herein, a dose of 1 mg/kg body for each active compound represents an amount of each active agent such that in a dose of 1 mg/kg the amount of curcumin compound applied to a subject weighing 70 kg is 70 mg, and the amount of 6-gingerol compound is also 70 mg. Doses of the curcumin and gingerol compounds are not necessary the same; e.g., the composition may be formulated so that application of a set amound provides a dose of 1 mg/kg of curcumin and 0.5 mg/kg of gingerol. A suitable dose of each of the active agents can however be from about 0.001 to about 0.1 mg/kg body weight, such as about 0.01 to about 0.050 mg/kg body weight. Doses from about 1 to 100, 200, 300, 400, and 500 micrograms are appropriate. As noted herein, repeat applications are contemplated. Repeat applications are typically applied from about two times per day to about once per week, or when the skin is or will be prone to abrasion and other injury or trauma such as extended photodamage and corticosteroid use.

Still other dosage levels of each of the active agents curcumin and 6-gingerol can range between about 1 nanogram (ng)/kg and about 1 mg/kg body weight per day are contemplated. The dosage of each of the active agents will generally be in the ranges of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 100 mg per kg body weight, about 0.01 mg to about 10 mg per kg body weight, or about 0.1 mg to about 1 mg per kg body weight.

In some embodiments, the dosage of each of the active agents will generally be in the range of about 0.001 mg to about 0.01 mg per kg body weight, about 0.01 mg to about 0.1 mg per kg body weight, about 0.1 mg to about 1 mg per kg body weight, or about 1 mg per kg body weight. If more than one curcumin and more than one gingerol are used, the dosage of each active agent curcumin and gingerol need not be in the same range as the other. For example, the dosage of one curcuminoid analog can be between about 0.001 mg to about 1 mg per kg body weight, and the dosage of another curcuminoid analog can be between about 0.001 mg to about 1 mg per kg body weight. The same applies to gingerol compounds, such as 6-gingerol, 8-gingerol, 10-gingerol, 6-paradol, 6-shagaol and cassumunin. As noted herein, repeat applications are contemplated. Repeat applications are typically applied about twice per day to about once per week.

Quantities of the present skin augmentation compositions that can be typically applied per application are, in mg composition/square centimeter (cm²) skin, ranging from about 0.01 mg/cm² to about 10 mg/cm². A particularly useful application amount is about 1 mg/cm² to about 2 mg/cm². Other useful doses for each of the active agents can range from about 1 microgram to about 1 milligram of each active agent/cm² of skin. Certain doses will be about 1-2, about 1-5, about 2-4, about 5-7, and about 8-10 micrograms of each active agent/cm² of skin. Other useful doses are greater than about 10 micrograms of each active agent/cm² of skin size, including about 15 micrograms/cm² of skin size, about 20 micrograms/cm² of skin size, about 25 micrograms/cm² of skin size, about 30 micrograms/cm² of skin size, about 35 micrograms/cm² of skin size, about 40 micrograms/cm² of skin size, about 50 micrograms/cm² of skin size, and about 100 micrograms/cm² of skin size. Other useful doses are about 150 micrograms/cm² of skin size, about 200 micrograms/cm² of skin size, about 250 micrograms/cm² of skin size, or about 500 micrograms/cm² of skin size. As noted herein, repeat applications are contemplated. Repeat applications are typically applied about twice per day to about once per week.

The active agents of the present technology can be administered separately at different times during the course of therapy, or concurrently in divided or single combination forms. In some embodiments, the skin augmentation composition useful for preventing and shortening the time required for wound healing is administered by topical administration (peripherally or directly to a skin site), including but not limited to topical administration using solid supports (such as dressings and other matrices) and medicinal formulations (such as gels, mixtures, suspensions and ointments). In one embodiment, the solid support comprises a biocompatible membrane or insertion into a skin treatment site. In another embodiment, the solid support comprises a dressing or matrix. In one embodiment of the invention, the solid support composition can be a slow release solid support composition, in which the active agents useful for skin augmentation is dispersed in a slow release solid matrix such as a matrix of alginate, collagen, or a synthetic bioabsorbable polymer. Preferably, the solid support composition is sterile or low bio-burden. In one embodiment, a wash solution comprising the skin augmentation composition in a cosmetically acceptable carrier can be used.

In some embodiments, the skin augmentation composition is provided in the form of a dressing or matrix, for example, dressings, gels, foams and the like. In some embodiments, the active agents of the present technology are provided in the form of a liquid, semi solid or solid composition for application directly, or the composition is applied to the surface of, or incorporated into, a solid contacting layer such as a dressing gauze or matrix. The dressing composition can be provided for example, in the form of a fluid or a gel. The active agents can be provided in combination with conventional pharmaceutical excipients for topical application.

In some embodiments, the skin augmentation composition can be added to suitable contact layer dressings, including, for example, thin, non-adherent sheets placed on an area to protect tissue from for example, direct contact with other agents or dressings applied to the treatment site. In some embodiments, contact layers can be deployed to conform to the shape of the area of the skin treatment site and are porous to allow the skin augmentation composition to pass through for absorption onto the skin treatment site.

Elastic Bandages: suitable elastic bandages can include dressings that stretch and conform to the body contours. In certain embodiment, the fabric composition can include for example, cotton, polyester, rayon or nylon. In certain other embodiments, the elastic bandage can for example, provide absorption as a second layer or dressing, to hold a cover in place, to apply pressure or to cushion a treatment site.

Foams: suitable foam dressings can include sheets and other shapes of foamed polymer solutions (including polyurethane) with small, open cells capable of holding liquid solution of the skin augmentation composition. Exemplary foams can be for example, impregnated or layered in combination with other materials. In certain embodiment, the absorption capability can be adjusted based on the thickness and composition of the foam. In certain other embodiments, the area in contact with the treatment site can be non-adhesive for easy removal. In yet another embodiment, the foam can be used in combination with an adhesive border and/or a transparent film coating that can serve as an anti-infective barrier.

Gauzes & Non-Woven dressings: suitable gauze dressings and woven dressings can include, dry woven or non-woven sponges and wraps with varying degrees of absorbency. Exemplary fabric composition can include cotton, polyester or rayon. In certain embodiments, gauzes and non-woven dressing can be available sterile or non-sterile in bulk and with or without an adhesive border. Exemplary gauze dressings and woven dressings can be used for moderate to slow release of the skin augmentation composition and covering a variety of wound treatment sites.

Hydrogels (Amorphous): suitable amorphous hydrogel dressings can include formulations of water, polymers and other ingredients with no shape, designed to donate moisture and to maintain a moist healing environments and or to rehydrate the skin treatment site while concomitantly releasing a therapeutically effective amount of the skin augmentation composition. In some embodiments, hydrogels can be used in combination with a secondary dressing cover.

Hydrogel Impregnated Dressings: suitable impregnated hydrogel dressings can include gauzes and non-woven sponges, ropes and strips saturated with an amorphous hydrogel. Amorphous hydrogels can include for example, formulations of water, polymers and other ingredients with no shape, designed to donate moisture to a dry treatment site and to maintain a moist healing environment while concomitantly releasing a therapeutically effective amount of the skin augmentation composition.

Hydrogel Sheets: suitable hydrogel sheets can include for example, three-dimensional networks of cross-linked hydrophilic polymers that are insoluble in water and interact with aqueous solutions by swelling. Exemplary hydrogels are highly conformable and permeable and can release varying amounts of the skin augmentation composition depending on their composition. In some embodiments, the hydrogel is non-adhesive against the skin treatment site or treated for easy removal. The released rate of the skin augmentation composition from the hydrogel can be adjusted depending on the chemical affinity of the hydrogel for the composition. Generally, the released composition provides an amount of each active agent in the range of about 0.01 mg/cm² to about 10 mg/cm² of skin treated.

The routes of administration and dosages described herein are intended only as a guide since a skilled physician will determine the optimum route of administration and dosage for any particular patient and skin treatment site.

A wide range of quantities of the skin augmentation composition of the present technology can be employed to provide an improved skin appearance and/or feel benefit, improved skin function and resistance to delayed healing after skin abrasion.

Regulating and augmenting the condition of the subject's skin can be practiced by applying a skin augmentation composition of the present technology in the form of a skin lotion, cream, gel, foam, ointment, paste, emulsion, spray, conditioner, tonic, cosmetic, after-shave, or the like that is preferably intended to be left on the skin for some esthetic, prophylactic, augmentative, therapeutic or other benefit (i.e., a “leave-on” composition). After applying the skin augmentation composition to the skin, it can be left on the skin for a period of at least about 15 minutes, or at least about 30 minutes, or at least about 1 hour, or for at least several hours, for example, up to about 12 hours.

Any part of the external portion of the subject's skin can be treated, e.g., face, lips, under-eye area; eyelids, scalp, neck, back, torso, arms, hands, legs, feet etc. In some embodiments, the skin augmentation composition of the present technology can be specifically applied to skin surface areas of the body that are chronically damaged or atrophied as a result of diabetes, photodamage and exposure to chronic corticosteroid use and on areas of the skin in which abrasion is likely to occur. The skin augmentation composition can be applied with the fingers or with an implement or device (e.g., synthetic or natural material, pad, cotton ball, applicator pen, spray applicator, and the like).

Another approach to ensure a continuous exposure of the skin to at least a minimum level of the beneficial skin augmentation composition is to apply the composition in a patch, for example, to selected tissues such as the arms, hands, feet, trunk and face. Such an approach is particularly useful for problem skin areas needing more intensive treatment (e.g., areas of skin likely to receive abrasions that can later turn into slow healing wounds, for example, the feet of diabetic patients). The patch can be occlusive, semi-occlusive or non-occlusive and can be adhesive or non-adhesive. The skin augmentation composition can be contained within the patch or be applied to the skin prior to application of the patch. The patch is preferably left on the skin for a period of at least about 5 minutes, or at least about 15 minutes, or at least about 30 minutes, or at least about 1 hour, or at night as a form of night therapy.

Example embodiments are provided so that this disclosure will be thorough, and will fully convey the scope to those who are skilled in the art. Numerous specific details are set forth such as examples of specific components, devices, and methods, to provide a thorough understanding of embodiments of the present technology. It will be apparent to those skilled in the art that specific details need not be employed, that the examples can be embodied by many different forms and that neither should be construed to limit the scope of the disclosure. In some example embodiments, well-known processes, well-known device structures, and well-known technologies are not described in detail.

### Examples

### Example 1

### Wound Healing in Curcumin and Gingerol Treated Rats

Materials and Methods

The preparation of curcumin used here was obtained from LKT Laboratories, Inc. (St. Paul, Minn.). The concentration of curcumin in the preparation was 93.6% with 5.7% other curcuminoids as determined by HPLC.

6-gingerol—enriched ginger extract: The ginger extract used here is a yellow-green, oily substance obtained as a CO₂ extract from pulverized ginger roots (Zingiber officinale) (Dalton Chemical Laboratories; Toronto, Ontario, Canada). 6-gingerol constitutes approximately 32% of the extract on a per weight basis as determined by HPLC.

The botanicals were dissolved in a vehicle (70% ethanol and 30% propylene glycol) at 10.0% for curcumin and 3% for the ginger extract. The concentrations chosen for the two botanicals were based on results from preliminary in vitro studies and in vivo studies with healthy hairless rats.

Experimental Models

Rat Model: Hairless rats were treated for 21 days topically over the back and flank with 500 μL of solutions containing 10% curcumin, 3% ginger extract or the combination of the two. Vehicle alone served as control. During the treatment phase, animals were examined daily for changes in the gross appearance of the skin. After the initial 21-day pretreatment period, rats were treated with Temovate® (clobetasol propionate) cream along with the botanicals for an additional 15 day period. Temovate® (0.05% solution of clobetasol propionate in cream base) was obtained from Glaxo Smith Kline (Philadelphia, Pa.). Temovate® was applied in the morning and botanicals were applied in the evening. At the end of the treatment period, abrasion wounding was performed. Briefly, under general anesthesia (ketamine/xylazine), paravertebral skin from the back and flanks was cleaned with 70% ethanol. A pre-measured circular area, approximately 6 cm in diameter, was scrubbed with a stiff-nylon bristle brush lightly wetted with acetone and concomitantly abraded with a piece of course sanding sponge. Abrasion was sufficient to remove the thin epidermis and the upper most part of the subepithelial stroma. Oozing of the fluid (with a small amount of blood) into the abraded area indicated that the appropriate degree of abrasion had been achieved. The degree of injury was designed to approximate abrasions that commonly occur after a minor scrape.

Wounding was performed under sterile conditions in a laminar flow hood. Wound size was determined daily by measuring the X-axis and Y-axis of the scabbed wound and calculating the area of the remaining scab. At the time of wound closure, animals were sacrificed and duplicate 6 mm skin punches were collected from wounded and healed skin. One biopsy was fixed in 10% buffered formalin and used for histological analysis. The other biopsy was put in organ culture for 3 days. Briefly, the skin was cut into pieces of approximately 2 mm on a side, and four-five such pieces were incubated for 3 days in 0.5 mL of a culture medium consisting of growth factor-free, serum-free, Keratinocyte Basal Medium (KBM) (Lonza, Walkersville, Md.). Before use, the culture medium was supplemented with Ca²⁺ to a final concentration of 1.4 mM. At the end of the 3-day incubation period, the organ culture fluid was collected and assayed for soluble type I collagen by western blotting and MMP-2 and -9 gelatin zymography (see below).

Analysis of Type I Collagen: Organ culture fluids were assayed for type I collagen by Western blot analysis. Briefly, organ culture fluids representing an equal quantity of protein were resolved using 8% SDS-PAGE under non-reducing conditions and transferred to nitrocellulose membranes. The membranes were blocked with 5% non-fat milk solution in Tris-buffered saline with 0.1% Tween (TTBS) for 1 hour at room temperature. Following this, they were incubated overnight with a rabbit antibody to rodent type I collagen (1:10,000 dilution) (Abcam Inc., Cambridge, Mass.) in the same buffer at 4° C. The membranes were then washed with TTBS and bound antibody was detected using the Phototope-HRP western detection kit (Cell Signaling Technologies, Inc., Danvers, Mass.). Images were scanned, digitized, and quantified using NIH image analysis software.

Analysis of Metalloproteinases: MMP-2 and MMP-9: Gelatin-embedded enzymography (zymography) was used to assess levels of latent and active MMP-2 and MMP-9 in organ culture fluids. SDS-PAGE gels were prepared with the incorporation of gelatin (1 mg/mL) at the time of casting. After electrophoresis under non-reducing conditions to separate proteins and overnight incubation to allow for substrate digestion, zones of hydrolysis were identified as “holes” in the stained gels and quantified. Values for latent and active MMPs-2 and -9 bands were obtained following digitization and quantification.

Statistical Analysis: Data were analyzed using one-way analysis of variance (ANOVA) followed by the Bonferroni posttest for selected pairs (GraphPad Prism version 4.00 for Windows, GraphPad Software, San Diego, Calif.). For experiments in which there were only two groups, the Student t-test was used assess statistical significance of the differences. Data were considered significant at p<0.05.

Results

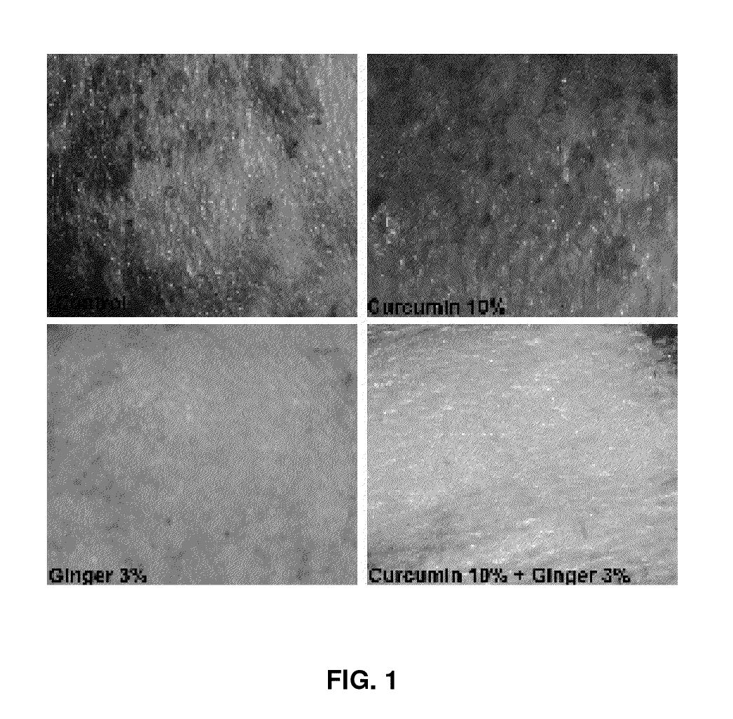

Topical treatment with curcumin and ginger extract increases type I collagen and decreases MMP-9 in the skin of healthy hairless rats. Based on in vitro experiments in which a wide range of concentrations of both curcumin and the ginger extract were tested, healthy hairless rats were treated topically once daily for 14 days with solutions containing 10% curcumin and 3% ginger extract, alone and in combination. Control rats were treated with vehicle alone. Animals were monitored closely during the treatment period of any visible changes in gross appearance of the skin. FIG. 1 demonstrates the appearance of the skin from animals treated for 15 days with vehicle alone or with the combination of 10% curcumin and 3% ginger extract. No irritation was observed with either agent alone or in combination. Observations included a reduction in mottling, seen with the ginger extract (with or without curcumin) and discoloration of the skin with curcumin. At the end of the treatment period, skin from the treated site of each animal was obtained. The skin was incubated in organ culture for 3 days. At the end of the incubation period, organ culture fluids were collected and analyzed for soluble type I collagen as well as for MMP-2 and MMP-9. Organ culture fluid from skin sites treated with curcumin and from skin sites treated with the combination of curcumin and ginger extract had increased type I collagen and decreased MMP-9 as compared to control skin organ culture fluid (not shown). There was also a decrease in MMP-2 but it was not as substantial as the decrease in MMP-9. Based on these findings, subsequent wounding studies were conducted with 10% curcumin and/or 3% ginger extract.

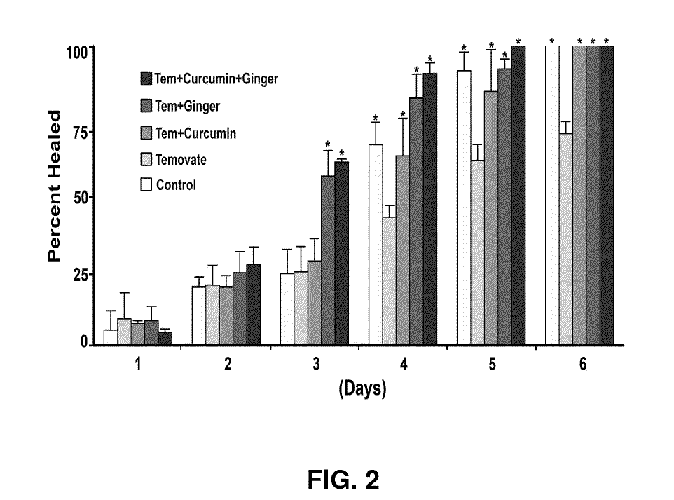

Topical pretreatment with curcumin and ginger extract improves abrasion wound healing in skin of corticosteroid-treated hairless rats. In the next series of experiments, hairless rats were treated with curcumin and ginger extract (alone and in combination) for 21 days. Following this, animals were treated once daily for an additional 15 days with a cortico steroid (Temovate®; i.e., clobetasol propionate). Treatment with the curcumin and ginger extract preparations were continued. Hairless rats that had been treated with vehicle alone during the initial 21-day period were also treated daily with the cortico steroid. These animals continued to receive vehicle as a control. At the end of the treatment phase, abrasion wounds were induced in all animals and the percent of wound healed was recorded. Three independent experiments with three animals in each group were conducted and the pooled data are presented in FIG. 2. Wound closure in the corticosteroid-treated animals was slower than in the non-steroid treated control animals. Wound closure was improved in those steroid-treated rats that also received either curcumin or ginger extract alone as compared to vehicle alone. Animals treated with the combination of curcumin and ginger showed the fastest healing rates.

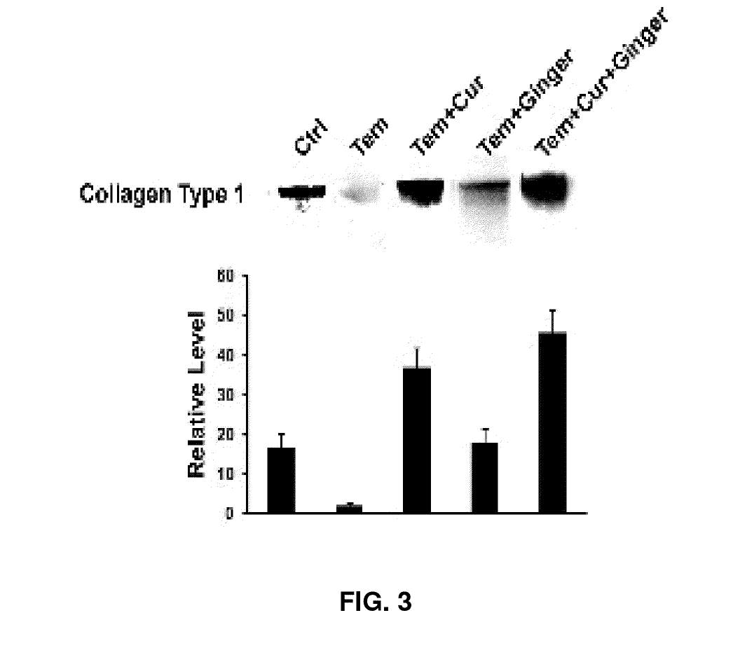

At the time of wound closure, skin from the center of the initial wound (most recently healed skin) was obtained and incubated in organ culture for three days. Organ culture fluids were collected at the end of the incubation period and analyzed for type I collagen and MMP-2/MMP-9. As seen in FIG. 3, type I collagen was reduced in the culture fluid from skin of rats treated with Temovate® as compared to control animals. This is consistent with other results demonstrating steroid-inhibition of collagen production in the skin following topical treatment. Rats treated with curcumin and/or ginger extract prior to corticosteroid treatment (and during steroid treatment) demonstrated elevated levels of type I collagen as compared to rats treated with Temovate® and vehicle. Of interest, collagen levels were higher following curcumin treatment (along with Temovate®) than were collagen levels in control culture fluids (from rats not exposed to Temovate®). The ginger extract alone also increased type I collagen levels in skin from Tenovate-treated rats, but not as effectively as curcumin alone or curcumin and ginger extract together.

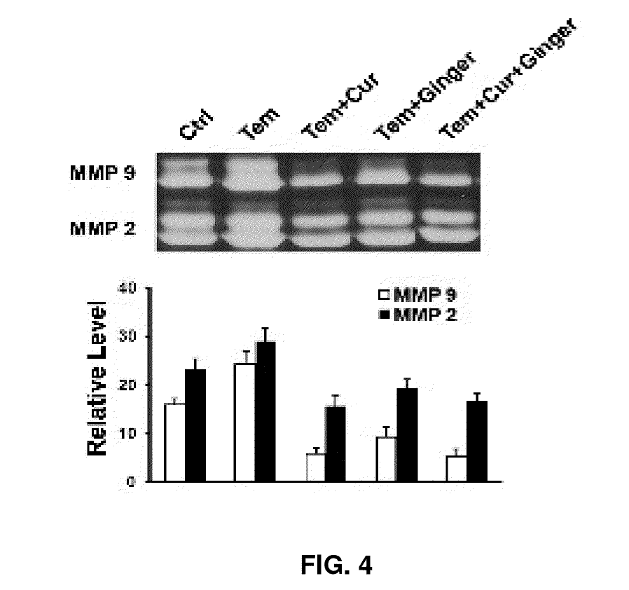

Gelatin zymography data are presented in FIG. 4. It can be seen from FIG. 4 that levels of both MMP-2 and MMP-9 were elevated in organ culture fluid of skin from Temovate®-treated rats relative to levels in control rat skin organ culture. Levels of both enzymes were reduced by curcumin and/or ginger treatment. As expected, MMP-9 was more sensitive to modulation than was MMP-2.

Discussion

The use of curcumin and ginger extract in a mixture for topical delivery is believed to overcome the limitations of therapeutics that are currently available. In the present study, topical pre-treatment of rats with a combination of the two natural products improved healing of subsequently-induced skin wounds in corticosteroid-treated rats. Each agent alone also facilitated improved wound healing, but the two in combination was more effective than either agent alone. No irritation was observed in the treated animals at any time during the pretreatment or wounding/wound-healing phase.

Unexpectedly, the two active agents work to provide synergistic improvement. Curcumin can increase collagen production, and the present experiments demonstrate this in corticosteroid-treated rats. Treatment of rats with the ginger extract alone had only modest effects on collagen levels and MMP levels. On the other hand, ginger is a source of potent anti-oxidants, and other studies have demonstrated improved skin wound healing in rats treated with other anti-oxidant moieties; e.g., lipoic acid. How anti-oxidants function in wound-repair is, itself, not fully understood. Inflammation is a normal part of the wound-healing process. However, the inflammatory response tends to be exaggerated in non-healing wounds. Although not wishing to be bound by theory, suppressing oxidant function may help, therefore, by reducing pro-inflammatory oxidant effects. Alternatively, metabolically-activated cells, themselves, generate a large amount of pro-oxidants. It is possible that the anti-oxidant activity associated with the ginger extract serves to counteract the pro-oxidant activity in cells metabolically-activated by curcumin.

The ginger root is also a source of multiple biologically active gingerol compounds, including 6-gingerol, 8-gingerol, 10-gingerol, 6-paradol, 6-shagoal and cassumunin. 6-gingerol is the most abundant analogue in the extract used here, and represents approximately 32% of the total extract. However, 6-gingerol (purified to greater than 95%) appears to have the same in vitro activities as the crude extract itself. In addition, synthetic curcuminoids and synthetic gingerols are also available. One goal is to have a preparation that is inexpensive to prepare so that individuals at risk for non-healing skin ulcers can utilize it on a long-term basis as a wound preventative.

### Example 2

### Synergistic Effects of the Combined Active Agents in a Human Model of Skin Repair

The data summarized here are from studies with human dermal fibroblasts and human skin in organ culture. Three sets of data are included. One set of data demonstrates that the combination of curcumin and ginger extract can preserve viability of human dermal fibroblasts maintained in monolayer culture under conditions in which cell survival does not occur in the absence of the two agents. The other two sets of data show that the combination of ginger and curcumin compounds suppresses the elaboration of matrix metalloproteinase-1 (MMP-1) in monolayer cultures of human dermal fibroblasts and in human skin organ culture. These data show, specifically, that ginger extract alone actually raises MMP-1 levels. However, when curcumin is provided along with the ginger extract, enzyme induction is strongly suppressed.

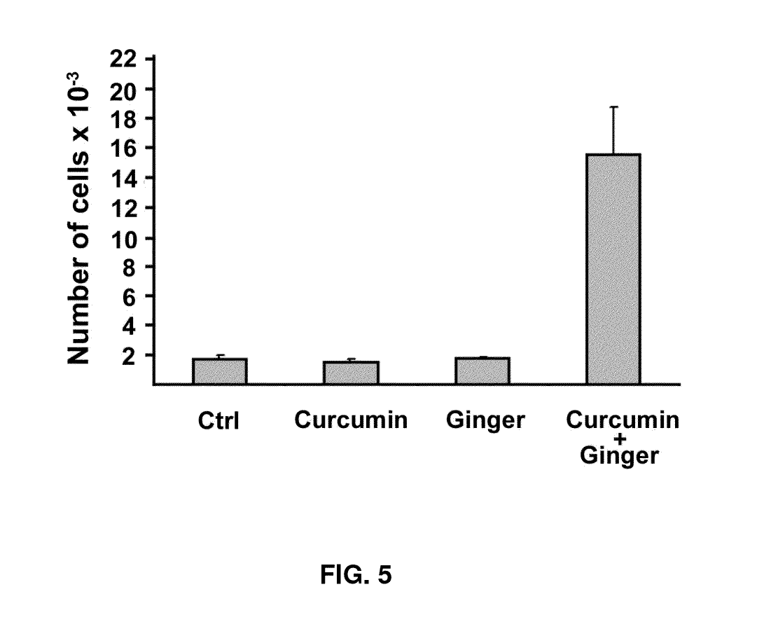

There are several components to healthy skin. One is the vasculature. It was unexpectedly found that the ginger component increased small vessel density in the treated skin. This is critical to healthy skin function. Another component is the rebuilding of damaged collagen. Curcumin is a potent builder of new collagen. It also seems to inhibit the excess MMPs that are produced in response to the ginger component. The combination of the two active agents when applied or administered to skin, one observes an improved connective tissue and increased vasculature. Thus, the two agents act on different aspects of the skin-repair process and therefore act synergistically. For example, as shown in FIG. 5, the number of fibroblasts preserved is synergistically increased when the two active agents curcumin and ginger extract are co-administered. Furthermore, one of the components (curcumin) mitigates an unwanted consequence (excess MMP production) occurring in response to the gingerol active.

FIG. 5 shows that combinations of curcumin and ginger extract preserve viability of human dermal fibroblasts. Fibroblasts were incubated under control conditions (KBM culture medium) or in the same culture medium supplemented with 2.5 μM curcumin, 0.5 μg per mL ginger extract or the combination of the two. The combination of the curcumin and ginger extract resulted in a synergistic and significant increase in cell number over that of the control.

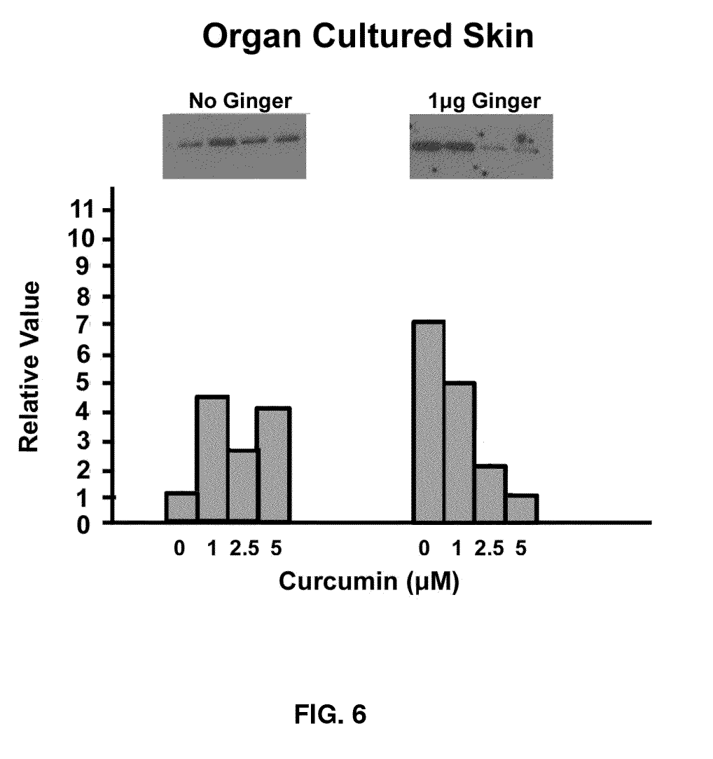

FIG. 6 shows MMP-1 levels in human skin organ culture fluid. Human skin was incubated in organ culture under control conditions (Ca²⁺—supplemented KBM culture medium) or in the same culture medium with 1 μg per mL ginger extract. Increasing amounts of curcumin were added. As can be seen from FIG. 6, the ginger extract induced MMP-1 expression and this was inhibited by curcumin.

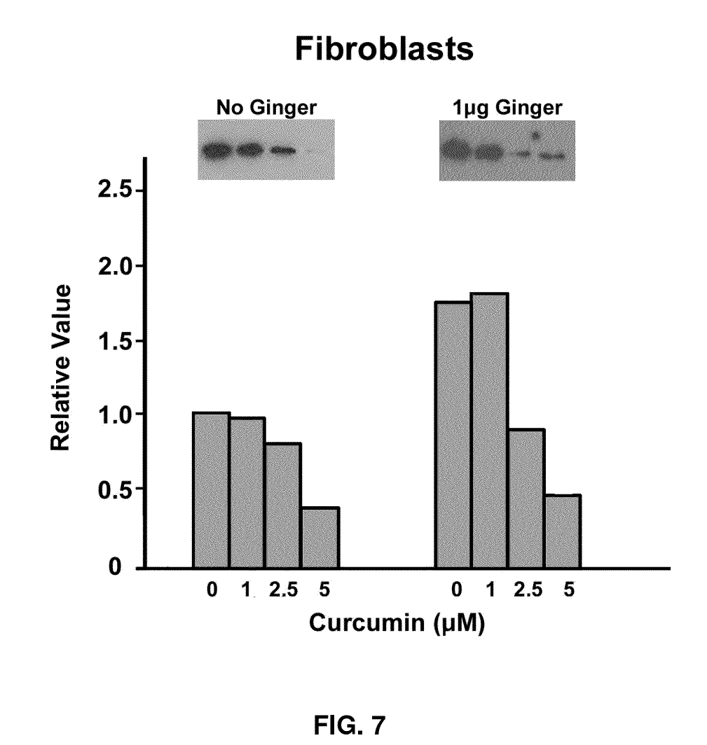

As shown in FIG. 7, MMP-1 levels in fibroblast culture fluid were measured in cell cultures incubated with ginger and curcumin. Fibroblasts were incubated under control conditions (Ca²⁺—supplemented KBM culture medium) or in the same culture medium supplemented with 1 μg per mL ginger extract. Increasing amounts of curcumin were added. As can be seen from the FIG. 7, the ginger extract induced MMP-1 expression and this was inhibited by curcumin.

The synergistic effects of combination of curcumin with 6-gingerol found in ginger extract explains three important findings. First, the findings provide clear evidence of a synergistic interaction between curcumin and ginger extract. Second, the studies were conducted with human dermal fibroblasts and human skin in organ culture. This provides evidence that what was observed in the rat model will be applicable to human skin, as well. Third, promoting skin cell viability is of value since the processes that lead to damaged skin result in fewer viable cells in the dermis. In regard to MMP-1, this is the major collagen—degrading enzyme in the skin. Thus, the findings here support the use of the combination of curcumin and gingerol compounds to affect skin repair.

The combination of curcumin and ginger extract function together to promote collagen building. The ginger compound serves to raise the level of collagen-degrading enzymes, such as matrix metalloproteinases (MMPs) as one of its functions. While the curcumin compound suppresses the MMP elevation stimulated by the ginger compound. This is observed in the rat model with MMP-9 and is observed in the human skin organ culture with MMP-1.

The embodiments and the examples described herein are exemplary and not intended to be limiting in describing the full scope of compositions, systems, and methods of the present technology. Equivalent changes, modifications and variations of some embodiments, materials, compositions and methods can be made within the scope of the present technology, with substantially similar results.

1.-9. (canceled)

10. A method for augmenting skin, the method comprising administering to the skin a composition comprising 1% to 20% (w/v) curcumin, 0.1% to 10% (w/v) 6-gingerol, and a carrier suitable for topical application wherein the carrier comprises an emollient.

11. The method of claim 10, wherein the skin is skin damaged from corticosteroid use.

12. The method of claim 10, wherein the composition is applied to the skin to augment the skin for wound repair.

13. The method of claim 12, wherein the composition is administered prior to the wound.

14. The method of claim 12, wherein the wound is a skin abrasion.

15. The method of claim 12, wherein the wound is a surgical incision.

16. The method of claim 12, wherein the wound is the recipient site of a skin graft.

17. The method of claim 12, wherein the wound is the donor site for a skin graft.

18. The method of claim 10, wherein the composition is applied to augment chronologically aged skin.

19. The method of claim 10, wherein the composition is applied to augment diabetic skin.

20. The method of claim 10, wherein the composition is applied to augment photoaged skin.

21. A method for augmenting skin, the method comprising administering to the skin a first composition comprising curcumin and a carrier suitable for topical application comprising an emollient and administering a second composition comprising 6-gingerol and a carrier suitable for topical application comprising an emollient.

22. The method of claim 21, wherein administration of one of the first and second compositions is followed by administration of the other one of the first and second compositions in less than about a day.

23. The method according to claim 10, wherein the composition comprises ginger extract.

23. The method according to claim 10, wherein the composition comprises turmeric extract.

23. The method according to claim 10, wherein the composition comprises ginger extract and turmeric extract.
